SEQUENCE LISTING (3 sequences)

SEQ ID NO: 1 - DNA, 29 bp, Artificial Sequence
  Primer
ttttttttttgaatgatatgcgacgcgtg

SEQ ID NO: 2 - DNA, 29 bp, Artificial Sequence
  Primer
ttttttttttcacgcgtcgcatatcattc

SEQ ID NO: 3 - DNA, 29 bp, Artificial Sequence
  Primer
ttttttttttcatgcatcgtatgtcgtaa

(19) United States
(12) Patent Application Publication
(10) Pub. No.: US 2013/0237450 A1
(43) Pub. Date: Sep. 12, 2013

(54) Method for Detecting Nucleic Acids

(71) Applicant: Bernhard Ronacher, Linz (AT)

(72) Inventor: Bernhard Ronacher, Linz (AT)

(73) Assignee: ANAGNOSTICS BIOANALYSIS GMBH, St. Valentin (AT)

(21) Appl. No.: 13/988,089

(22) PCT Filed: Nov. 18, 2011
(86) PCT No.: PCT/EP2011/070449

§ 371 Date: May 17, 2013

(30) Foreign Application Priority Data
Nov. 19, 2010 (EP) 10191872.0

(51) Int. Cl. C12Q 1/68

(52) U.S. Cl. CPC C12Q 1/6837
USPC 506/9

(57) ABSTRACT

A method for simultaneously detecting at least one specific DNA target molecule in a plurality of samples by means of nucleic acid amplification is provided. The method comprising: bringing at least two DNA samples comprising DNA in contact with forward and reverse primers, which in the 3′ portion contain a nucleotide sequence that is complementary to the amplifying DNA target molecule, and which in the 5′ portion comprises an oligonucleotide having an artificial DNA sequence that is associated with the individual samples; amplifying the samples; contacting the amplified samples with a solid substrate on which oligonucleotides are immobilized at a preselected site, said oligonucleotides comprising at the 3′ ends the artificial DNA sequence associated with the individual samples; and ascertaining binding of the amplification products at the oligonucleotides immobilized on the solid substrate.

The present invention relates to methods for simultaneously detecting at least one specific target DNA molecule in at least two samples.

In different hybridization methods that are based on the formation of DNA double strands with complementary sequences, artificial DNA sequences may be used. These sequences usually comprise between 10 and 25 nucleotides and are not homologous to any of the known natural sequences. These oligonucleotides can comprise a second sequence portion. The portion located at the 5′ end is the artificial portion, whereas the portion located at the 3′ end is the natural portion that is complementary to the target sequence in the sample. If oligonucleotides designed in such a manner are used in a polymerization reaction, the amplification products thus obtained can unambiguously be assigned via the sequence of the oligonucleotides incorporated at the 5′ end. In a subsequent hybridization of the amplification products thus obtained, oligonucleotides are used at the solid phase that are complementary to the artificial sequences in the amplification product. In this manner it is possible to detect, e.g., point mutations subsequently to a primer extension (see for example DE 3807994, US 2003/148284, EP 1 186 669 and EP 0 530 794). The four primers used therein differ from one another at the 3′ end and carry an A, T, G or C at the 5′ portion of the primer as each of them carries an entirely different artificial sequence. This renders mutation analyses relating to a plurality of DNA base pairs a technical impossibility. This is due to the use of very high primer concentrations with a very similar primer sequence. (For instance, for a triplet: primers for the first base [a](four) for the second base [b] (4*4) and for the third base [c] (4*4*4), i.e. a total of 4³=64 primers in the solution would have to be used. In known multiplex PCR systems, the maximum number is currently lower than 30.

WO 2005/042759 describes a method for the determination of a gene expression profile. In all analyses of such profiles RNA molecules are used that are transcribed into DNA molecules using a reverse transcriptase. According to WO 2005/042759, the DNA molecules thus generated are amplified with a plurality of primers coding for both a template-specific sequence and a bar code sequence, by means of which it is possible to determine whether or not the analyzed sample expresses a specific gene.

The hitherto known methods for the detection of specific molecules in a sample comprise the steps of sampling, sample preparation, target molecule enrichment (optionally including labeling), hybridization to the solid phase, washing and a detection reaction (e.g. determination of color). In this process, a hybridization unit (e.g. a glass plate) is required. For each next sample to be analyzed, a fresh hybridization unit has to be used (or extensive regeneration steps have to be performed). One drawback of such methods lies in the very low throughput (samples hybridized per time unit). A simultaneous processing cannot be conducted with such methods.

It is therefore an object of the present invention to provide methods that enable the specific detection of molecules, in particular nucleic acid molecules, in a sample.

Thus, the present invention relates to a method for simultaneously detecting at least one specific target DNA molecule in a plurality of samples by means of nucleic acid amplification, comprising the following steps:
a) providing at least two samples comprising DNA,
b) contacting the samples from step a) with a forward primer and a reverse primer comprising a 3′ and a 5′ portion, wherein the forward and the reverse primer each have a nucleotide sequence in the 3′ portion that is complementary to the DNA target molecule to be amplified and the forward or the reverse primer comprises in the 5′ portion thereof an oligonucleotide having an artificial sequence which is associated with the individual samples,
c) amplifying the samples from step b),
d) contacting the amplified samples from step c) with a solid support on which oligonucleotides are immobilized at a preselected site, wherein said oligonucleotides comprise at the 3′ end thereof the artificial sequence which is associated with the individual samples, and
e) detecting the binding of the amplification products from step c) to the oligonucleotides immobilized on the solid support.
   With the method according to the present invention it is possible to simultaneously determine a plurality of specific DNA molecules in a plurality of samples within one single detection system and one single method step. In a first method step, the DNA molecules to be detected in the samples are amplified. One of the primers used (the forward or the reverse primer) has in the 5′ portion thereof an artificial nucleic acid sequence that is not present in any of the samples or is not capable of generating an amplification product with the further primer under amplification conditions. The artificial nucleic acid sequence is specific for a respective sample, i.e. one specific sequence is associated with each sample. In this way, the products generated in the amplification process having the artificial nucleic acid sequence at the 5′ end and the complementary artificial nucleic acid sequence at the 3′ end of the complementary strand can be bound to the solid support on which oligonucleotides comprising the artificial nucleic acid sequence are immobilized. If the sample thus comprises the target sequence, the amplification products will bind to the solid support.

These method steps distinguish the method according to the present invention from the methods known in the art. For instance, WO 2005/042759 describes a method that is apparently suitable for determining the gene expression profile of an organism. The method employs primers that, in contrast to the primers according to the present invention, are complementary to a plurality of different genes. In addition, these gene-specific primers have a sequence region (bar code sequence) that is identical in all primers employed. Once the amplification of all genes of the expression profile is completed, the amplification products are immobilized on a solid support which comprises oligonucleotides having sequences that are complementary to all genes that are theoretically expressed in the organisms to be analyzed and for which corresponding primers were designed. According to WO 2005/042759, the amplification products immobilized on the solid support are detected via the so-called bar code sequence. As the location of specific oligonucleotides on the solid support is known, it can thus be determined whether a specific gene, which has been associated with the oligonucleotide on the solid support, is expressed or not expressed in the respective organisms. The method according to WO 2005/042759 is neither intended for conducting a method for simultaneously detecting at least one specific DNA target molecule in a plurality of samples, nor would the method according to WO 2005/042759 be suitable for such a purpose. On the one hand, the method according to WO 2005/042759 is merely suitable for the determination of those amplification products bound to a solid support whose sequences are obviously different. However, with this method it is not possible to detect whether the amplification products originate form different starting materials as the only fact that can be determined is the binding of the amplification product to a specific oligonucleotide on a solid support, while the origin of the amplification product cannot be determined. This is only possible by means of a parallel amplification of artificial DNA sequences which are associated with the individual samples, as is the case in the method according to the present invention.

The step of contacting the amplified sample with the solid support is performed under conditions that allow for specific binding of the amplification products to the oligonucleotides that are immobilized on the solid support. In order to increase the efficiency of this binding and to remove non-specifically bound nucleic acid molecules from the solid support, the solid support can be washed after the contacting step. The washing steps and the steps in which the amplification products are bound to the immobilized oligonucleotides are preferably conducted under stringent conditions.

According to the present invention, the term “stringent conditions” comprises conditions under which a DNA sequence will preferably hybridize to its target sequence and to a lesser extent or not at all to other sequences. In general, stringent conditions are selected such that the temperature is about 5° C. below the thermal melting point (Tₘ) for the specific sequence at a defined ionic strength and a defined pH value. The Tₘ represents the temperature (with defined ionic strength, pH value and nucleic acid concentration) at which 50% of the molecules that are complementary to the target sequence will hybridize to the target sequence in a state of equilibrium. Typically, stringent conditions are conditions under which the salt concentration corresponds to a sodium ion (or any other salt) concentration of at least about between 0.01 and 1.0 M at a pH value of between 7.0 and 8.3 and the temperature is at least 30° C. for short molecules (e.g. comprising between 10 and 50 nucleotides). In addition, stringent conditions can be achieved by the addition of destabilizing agents, such as formamide.

Suitable stringent hybridization conditions are also described, e.g., in Sambrook et al. (Molecular Cloning: A Laboratory Manual, 3rd edition, Cold Spring Harbor Laboratory Press, Cold Spring Harbor, N.Y.). Thus, the hybridization reaction may be, e.g., performed under the following conditions:

Hybridization buffer: 2×SSC, 10×Denhardt's solution (Ficoll 400+PEG+BSA; in a ratio of 1:1:1), 0.1% SDS, 5 mM EDTA, 50 mM Na₂HPO₄, 250 μg/ml herring sperm DNA; 50 μg/ml tRNA or 0.25M sodium phosphate buffer pH 7.2, 1 mM EDTA, 7% SDS, at a hybridization temperature of between 65° C. and 68° C.

Washing buffer: 0.2×SSC, 0.1% SDS at a washing temperature of between 65° C. and 68° C.

According to the present invention, the first steps until step c) of the method are conducted separately, i.e. individually for each sample. Subsequently to step c), the amplification products that have been ‘labeled’ with the artificial nucleic acid sequences that are associated with the individual samples can be mixed and their presence can be determined in parallel.

According to the present invention, at least two (preferably at least three, four, five, six, seven, eight, nine or ten) independent samples are processed separately. The nucleic acids (DNA or RNA) are separately labeled for the hybridization reaction (PCR). Thus, at least two identical processes are conducted in parallel which differ in that the primers employed all have identical portions at the 3′ end (for the target gene) and different oligonucleotide sequences at the 5′ end. Subsequently to the labeling reaction (PCR), the amplification products thus obtained are mixed and jointly transferred to a hybridization unit (solid support), where the formation of double strands with the oligonucleotides bound to the solid phase according to the transferred reverse complement sequences (artificial nucleic acid sequences) occurs. This leads to the conclusion that a successful hybridization to an oligonucleotide has to yield an amplification product and an amplification product is in turn only generated if a target (target gene) was contained in the sample. If there is no hybridization, the sample was negative. In this way it is possible to simultaneously analyze at least two independent samples in one single hybridization step, irrespective of whether the individual samples are positive or negative. This principle can be extended to “any” number of reactions conducted in parallel. For instance, it is possible to test 96 samples in a 96-well plate for a PCR event and to simultaneously hybridize and analyze the samples via the incorporated oligonucleotides on one solid support, whereby the throughput can be increased by a factor of 96.

According to the present invention, an “artificial nucleic acid sequence which is associated with the individual samples” or a “DNA sequence” is a DNA sequence which is not present in the sample or is not capable of binding to a DNA molecule contained in the sample under stringent conditions. In this manner it is facilitated that each amplified or labeled molecule in the sample can be bound to the solid support at a preselected site.

According to the present invention, the expression “at a preselected site” means that the oligonucleotides which are used in the present invention and bear the artificial DNA sequences which are associated with the individual samples are immobilized at at least one predefined location on the solid support according to sequence. When the oligonucleotides are spotted onto the solid support, each individual spot thus comprises only one type of oligonucleotide which is associated with one of the samples.

The solid support on which the oligonucleotides are immobilized can be of any geometrical shape that is suitable for comparable detection methods. The use of solid supports as they are conventionally employed in hybridization reactions is preferred. According to a preferred embodiment of the present invention, the solid support is of a planar or axially symmetrical shape.

The use of axially symmetrical solid supports is particularly advantageous in the method according to the present invention. Thus, the axially symmetrical solid support preferably consists of a rotor which can be inserted into a sample container while leaving a radial annular gap, wherein the rotor has a circumferential surface on which the oligonucleotides are immobilized. Such solid supports are well known in the art and are described in more detail, e.g., in WO 03/100401 and WO 2007/041734.

According to a preferred embodiment of the present invention, the oligonucleotides with the artificial nucleic acid sequence which is associated with the individual samples have a length of between 15 and 30, preferably of between 18 and 25 and even more preferably of between 19 and 23 base pairs.

According to a further preferred embodiment of the present invention, the oligonucleotides with the artificial nucleic acid sequence which is associated with the individual samples have a melting point of between 50 and 70° C., preferably of about 60° C. Conventional calculation methods can be employed for designing such nucleic acid sequences. Corresponding methods are well known in the art.

Finally, in order to determine whether a specific DNA molecule (amplification product) is present in one or more of the samples, it has to be determined whether a nucleic acid molecule is bound to the solid support in the course of the execution of the method. According to a preferred embodiment of the present invention the reverse primers are labeled with a dye. Such a label facilitates the detection of the binding of a labeled amplification product to the solid support.

According to the present invention, it is alternatively also possible to label the amplification products with a dye subsequently to step c) or d). Modified nucleotides (e.g. fluorescence-labeled nucleotides) can be employed in the amplification reaction. Another possibility is the use of non-dyes, such as chemical groups (molecules), which in turn are targets of further interactions. One example is biotin which generates a measurable signal with labeled streptavidin. The dye may have properties of absorption in different ranges of the electromagnetic spectrum, preferably in the ultraviolet (UV), visible or infrared range. However, the dye may also act as a particle. Gold particles or other types of particles are known which exhibit an interference with electromagnetic waves. Also conceivable is the use of magnetic or paramagnetic beads.

According to a preferred embodiment of the present invention, the dye is selected from the group consisting of Alexa Fluor, in particular Alexa Fluor 350, Alexa Fluor 488, Alexa Fluor 430, Alexa Fluor 555, Alexa Fluor 610 and Alexa Fluor 647, Bodipy (boron dipyrromethene), in particular Bodipy 493/503, Bodipy R6G-X, Bodipy 530/550, Bodipy 558/568, Bodipy 564/570, Bodipy TMR-X, Bodipy 576/589, Bodipy 581/591, Bodipy TR-X, Bodipy 630/650, Bodipy 650/665 and Bodipy FL, Oregon Green, in particular Oregon Green 488, Oregon Green 500 and Oregon Green 514, FAM, FITC, fluoresceine, fluoresceine-dT, Rhodamine Green, TET, JOE, Yakima Yellow, HEX, CY3, TAMRA, Rhodamine Red, CY3.5, ROX, Texas Red, CY5, CY5.5, IRD700 and IRD800.

The amplification products bound to the solid support may also be subjected to a primer extension. In this procedure, a polymerization reaction occurs subsequently to the hybridization to the immobilized primers, which is comparable to a PCR conducted in a liquid environment. The enzyme required for this reaction (polymerase) is added during or after the hybridization reaction, as are the ingredients required for the reaction that are contained in the buffer (dNTPs, salts and stabilizers). Subsequently to the hybridization reaction, the solid support is first adjusted to stringent conditions and then to polymerization temperature (Taq polymerase at 72° C. to 74° C.). The polymerization of the immobilized primer will then proceed until the hybridized DNA molecule serves as a matrix (template). The primer extension is performed in order to gain more information, in addition to the hybridization, on the amplification product obtained and therefore on the sample components (sequence of the DNA).

Whether a primer extension has taken place or not can be determined in a subsequent melting curve analysis. The extended primers and the hybridized amplification product have a significantly higher melting temperature as their lengths differ by more than double. Thereby, a difference of between 10 and 20° C. can be achieved. Only hybridized primers will completely melt off until the melting temperature of the amplification product is achieved and do not generate a signal at the spot. Extended primers exhibit a residual signal strength, whereby a clear distinction can be made. In this manner it can, for instance, be determined whether a gene (DNA molecule) bearing the primer sequence is present in a sample and which base pair follows next in the sequence.

According to a preferred embodiment of the present invention, the extension of the primers is detected by determining alterations in the melt-off behavior (see above). A primer extension can be detected via an altered melt-off behavior of the bound PCR products and can serve for distinguishing extended and non-extended primers, optionally also independently of an artificial sequence.

By means of the extension of primers it is also possible to produce modified targets for a shotgun sequencing.

The method according to the present invention can also be used for the preparation of sample material (DNA) for a subsequent sequencing process. Shotgun sequencing is based on sequencing reactions of pieces of DNA (or also of amplification products) which bear a similar sequence at both ends. This sequence is not present in the original DNA and has to be incorporated in a previously conducted preparation step. Currently, an extensive method is applied for ligating so-called tags (short pieces of double-stranded DNA) to all sample DNA fragments. This step optionally also includes the introduction of nucleic acid sequences into the tags for labeling the samples. This ligation step is not directed and not precise, which results in that all DNA fragments of the samples bear tags and are equally introduced into the sequencing process. However, only the sequence data of a very small number of fragments are required or desired for the analysis of a sample. Owing to this, shotgun sequencing is very efficient with respect to the method of sequencing, but very inefficient in its accuracy, thus rendering it an altogether very inefficient current method.

By means of the method according to the present invention, the number of fragments is reduced to the required number of fragments (e.g. oncogenes), which is facilitated by the introduction of sample identification sequences. In this process, primers specific for the sample DNA are used for a primer extension. At their 5′ end these primers carry a tag sequence and an additional artificial nucleic acid sequence for distinguishing between the individual samples. If a primer is extended complementarily to the DNA fragment in the sample, a template for a further reaction is formed. A primer that is present in a liquid can then bind to this newly formed target and in turn be extended. This primer will also bear a tag and the artificial nucleic acid sequence. The product thus formed is a single-stranded DNA corresponding to a defined sample DNA fragment and bearing an artificial tag at both ends. Subsequently, the solid support is subjected to a washing step at a high temperature. Remaining on the solid support are only the covalently bound primers and the newly formed single-stranded DNA molecules having a higher melting temperature and bearing the tag. By means of an incubation conducted at 94° C., these nucleic acid molecules are also eluted from the solid support. The eluate thus obtained then serves as a starting product for the shotgun sequencing. With the method according to the present invention it is thus possible to reduce the number of fragments to those target genes that bear tags and the artificial nucleic acid sequences, thus enabling the simultaneous processing of a plurality of samples in only one sequencing cycle. Thus, the shotgun sequencing method is very efficient and very accurate.

An alternative, generalizing aspect of the present invention relates to a method for simultaneously detecting at least one specific molecule in a plurality of samples, comprising the following steps:
a) providing at least two samples,
b) contacting the samples from step a) with a binding partner which specifically binds to the at least one molecule to be detected, wherein the binding partner is coupled to an oligonucleotide having an artificial nucleic acid sequence which is associated with the individual samples,
c) contacting the samples from step b) with a solid support on which oligonucleotides that are complementary to the oligonucleotides coupled to the binding partner are immobilized at a preselected site, and
d) detecting the binding of the molecules to be detected in the sample from step c) to the oligonucleotides immobilized on the solid support.

The concept underlying the present invention is not only suitable for the detection of nucleic acid molecules in a plurality of samples. According to the present invention it is also possible to detect specific molecules, such as polypeptides and proteins (e.g. antibodies) simultaneously in a plurality of samples. In this process, the samples are individually mixed with binding partners of the molecules to be detected before being contacted with the solid support. The specific binding partners are labeled with or coupled to the oligonucleotides which are associated with the individual samples. These oligonucleotides serve for binding the molecules to be detected at a preselected site on the solid support which is associated with the respective sample.

Binding of a specific molecule to the solid support can, e.g., occur via the addition of a second binding partner which is capable of specifically binding to the molecule. The second binding partner is preferably labeled with a dye.

According to a preferred embodiment of the present invention, the solid support is of a planar or axially symmetrical shape.

The axially symmetrical solid support preferably consists of a rotor which can be inserted into a sample container while leaving a radial annular gap, wherein the rotor has a circumferential surface on which the oligonucleotides are immobilized.

According to a further preferred embodiment of the present invention, the oligonucleotides with the artificial nucleic acid sequence which is associated with the individual samples have a length of between 15 and 30, preferably of between 18 and 25 and even more preferably of between 19 and 23 base pairs.

Preferably, the oligonucleotides with the artificial nucleic acid sequence which is assigned to the individual samples have melting point of between 50 and 70° C., preferably of about 60° C.

According to a preferred embodiment of the present invention, the molecules that are immobilized on the solid support are contacted before step d) with a second molecule-specific binding partner that is labeled with a dye.

The dye is preferably selected from the group consisting of Alexa Fluor, in particular Alexa Fluor 350, Alexa Fluor 488, Alexa Fluor 430, Alexa Fluor 555, Alexa Fluor 610 and Alexa Fluor 647, Bodipy (boron dipyrromethene), in particular Bodipy 493/503, Bodipy R6G-X, Bodipy 530/550, Bodipy 558/568, Bodipy 564/570, Bodipy TMR-X, Bodipy 576/589, Bodipy 581/591, Bodipy TR-X, Bodipy 630/650, Bodipy 650/665 and Bodipy FL, Oregon Green, in particular Oregon Green 488, Oregon Green 500 and Oregon Green 514, FAM, FITC, fluoresceine, fluoresceine-dT, Rhodamine Green, TET, JOE, Yakima Yellow, HEX, CY3, TAMRA, Rhodamine Red, CY3.5, ROX, Texas Red, CY5, CY5.5, IRD700 and IRD800.

The present invention is further illustrated by the following Figures and Examples without being limited thereto.

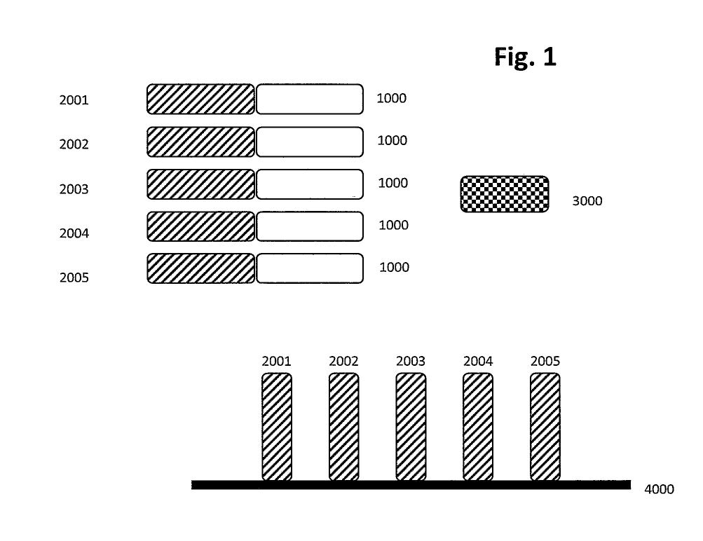

FIG. 1 shows an illustration of the basic principle of the method according to the present invention. In this case, samples obtained from 5 different patients are tested for the presence of a specific nucleic acid, wherein one artificial nucleic acid sequence is assigned to each individual patient. The forward primers employed (2000) comprise this artificial sequence (2001-2005) at the 5′ end. At the 3′ end, these primers have a portion that is capable of binding to the target nucleic acid molecule to be amplified (1000). The reverse primer (3000) is labeled with a dye and is identical for all individual reactions.

FIG. 1 further shows the surface of a solid support (4000) having immobilized thereon the oligonucleotides that comprise the artificial nucleic acid sequences which are associated with each patient. Each spot (2011-2055) comprises only one type of oligonucleotide, so that it is possible to directly associate one spot with one patient.

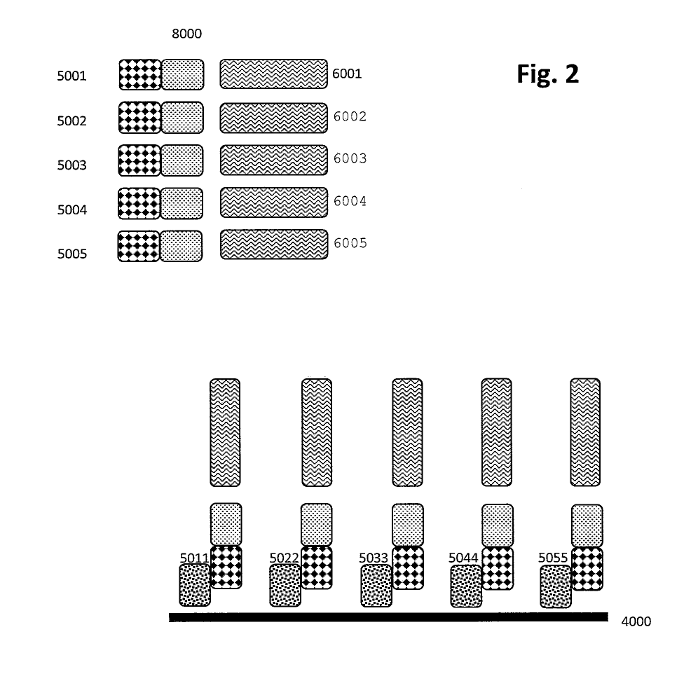

FIG. 2 shows an alternative aspect of the present invention. In this case, five samples comprising a molecule to be detected, e.g. an antibody (6001-6005), are independently contacted with respective binding partners (8000) to which sample-specific artificial oligonucleotides (5001-5005) are bound or coupled. These oligonucleotides are capable of binding to a solid support having immobilized thereon oligonucleotides (5011-5055) that have a nucleic acid sequence complementary to the oligonucleotides that are bound or coupled to the binding partners. As there are spots with only one type of oligonucleotide immobilized on the solid support, the molecules present in the sample will only bind to the predefined spot. The detection of the molecules bound to the solid support can, e.g., occur via a second binding partner that is labeled with a dye (secondary antibody).

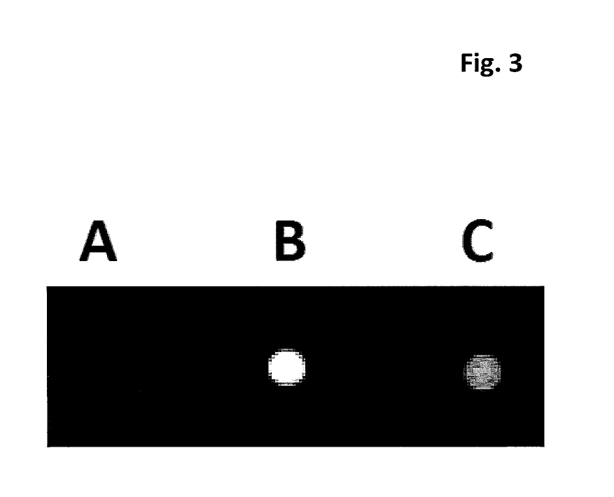

FIG. 3 shows a scan of three spots subsequently to a protein hybcode hybridization. A: oligonucleotide 572 (not complementary); B: directly printed protein and C: complementary oligonucleotide 571.

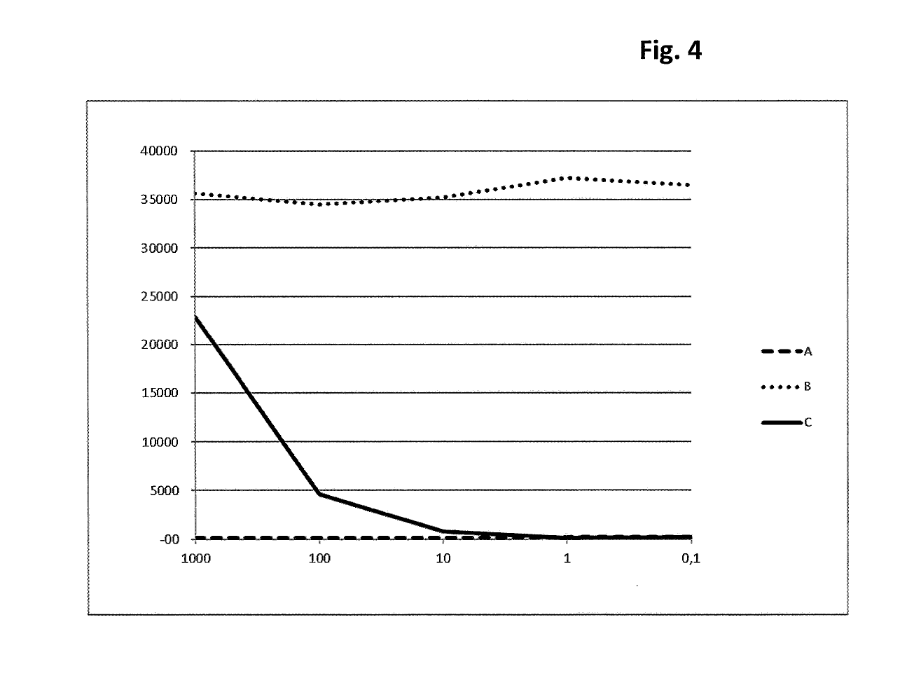

FIG. 4 shows a dilution series of an incubation of a protein-DNA oligonucleotide chimera with a subsequent incubation with anti-BSA antibody for detection (fluorescence-labeled). A: oligonucleotide 572 (not complementary); B: directly printed protein and C: complementary oligonucleotide 571.

### EXAMPLES

### Example 1

The chemical coupling among different biomolecules is already known in the art. One variant thereof is the covalent binding of protein and DNA (synthetic oligonucleotides). To this end, various chemical reactions may be employed. In a first step, the protein was chemically modified with a HyNic group (6-hydrazinopyridine-3-carboxylic acid). Simultaneously, a DNA oligonucleotide having an amino modification was chemically modified with a 4FB group (4-formylbenzamide). In a further chemical reaction, both groups were used to form a covalent bond which subsequently covalently links the protein to the DNA oligonucleotide. The uninvolved reaction partners were removed and the protein labeled in this manner was employed in the subsequent detection reactions.

In this series of experiments, BSA was used as a protein and the oligonucleotide 570 5′ TTTTTTTTTTGAATGATATGCGACGCGTG 3′ was used for the DNA codes.

Subsequently to the labeling reaction of the BSA with the oligonucleotide 570, the properties of the binding of the protein-DNA chimera to the reverse complementary oligonucleotide 571 5′ TTTTTTTTTTCACGCGTCGCATATCATTC 3′ and to a non-complementary oligonucleotide 572 5′ TTTTTTTTTTCATGCATCGTATGTCGTAA 3′ were tested. These oligonucleotides were immobilized on a solid support (e.g. Hybcell) as the array portion (row of spots). The incubation reaction comprised the following steps:
dissolving the protein-DNA chimera in incubation buffer consisting of PBS and 1% casein,
incubating the solid support for 15 min at 45° C. (i.e. 5° C. below the determined melting temperature of the coding sequence),
washing the solid support with PBS in order to remove unbound material,
incubating the solid support with fluorescence-labeled anti-BSA antibody,
washing the solid support with PBS in order to remove unbound material,
scanning the surface of the solid support in order to determine the signal intensities.
   This resulted in a clear discrimination of the different measurement points (spots) on the basis of the hybridization properties. Furthermore, the dependence on the concentration was measured and summarized in FIG. 4. This indicates that the use of the method according to the present invention is ideally suited for, inter alia, the assignment of signals in the simultaneous measurement of a plurality of samples with a solid phase array (e.g. Hybcell).

1. A method for simultaneously detecting at least one specific target DNA molecule in a plurality of samples by means of nucleic acid amplification, comprising the following steps:
a) providing at least two samples comprising DNA,
b) contacting the samples from step a) with a forward primer and a reverse primer comprising a 3′ and a 5′ portion, wherein the forward and the reverse primer each have a nucleotide sequence in the 3′ portion that is complementary to the DNA target molecule to be amplified and the forward or the reverse primer comprises an oligonucleotide in the 5′ portion that has an artificial sequence which is associated with the individual samples,
c) amplifying the samples from step b),
d) contacting the amplified samples from step c) with a solid support on which oligonucleotides are immobilized at a preselected site, wherein said oligonucleotides comprise at the 3′ end thereof the artificial sequence which is associated with the individual samples, and
e) detecting the binding of the amplification products from step c) to the oligonucleotides immobilized on the solid support.

2. The method according to claim 1, wherein the solid support is of a planar or axially symmetrical shape.

3. The method according to claim 2, wherein the axially symmetrical solid support consists of a rotor which can be inserted into a sample container while leaving a radial annular gap, wherein the rotor has a circumferential surface on which the oligonucleotides are immobilized.

4. The method according to claim 1, wherein the oligonucleotides with the artificial nucleic acid sequence which is associated with the individual samples have a length of between 15 and 30 base pairs.

5. The method according to claim 1, wherein the oligonucleotides with the artificial nucleic acid sequence which is associated with the individual samples have a melting point of between 50 and 70° C.

6. The method according to claim 1, wherein the reverse primers are labeled with a dye.

7. The method according to claim 1, wherein the amplification products are labeled with a dye subsequently to step c) or d).

8. The method according to claim 6, wherein the dye is selected from the group consisting of Alexa Fluor, in particular Alexa Fluor 350, Alexa Fluor 488, Alexa Fluor 430, Alexa Fluor 555, Alexa Fluor 610 and Alexa Fluor 647, Bodipy (boron dipyrromethene), in particular Bodipy 493/503, Bodipy R6G-X, Bodipy 530/550, Bodipy 558/568, Bodipy 564/570, Bodipy TMR-X, Bodipy 576/589, Bodipy 581/591, Bodipy TR-X, Bodipy 630/650, Bodipy 650/665 and Bodipy FL, Oregon Green, in particular Oregon Green 488, Oregon Green 500 and Oregon Green 514, FAM, FITC, fluoresceine, fluoresceine-dT, Rhodamine Green, TET, JOE, Yakima Yellow, HEX, CY3, TAMRA, Rhodamine Red, CY3.5, ROX, Texas Red, CY5, CY5.5, IRD700 and IRD800.

9. The method according to claim 1, wherein the amplification products bound to the solid support are subjected to a primer extension procedure.

10. The method according to claim 9, wherein the extension of the primers is detected by determining alterations in the melt-off behavior.

11. The method according to claim 4, wherein the oligonucleotides with the artificial nucleic acid sequence which is associated with the individual samples have a length of between 18 and 25 base pairs.

12. The method according to claim 4, wherein the oligonucleotides with the artificial nucleic acid sequence which is associated with the individual samples have a length of between 19 and 23 base pairs.

13. The method according to claim 5, wherein the oligonucleotides with the artificial nucleic acid sequence which is associated with the individual samples have a melting point of about 60° C.

14. The method according to claim 7, wherein the dye is selected from the group consisting of Alexa Fluor, in particular Alexa Fluor 350, Alexa Fluor 488, Alexa Fluor 430, Alexa Fluor 555, Alexa Fluor 610 and Alexa Fluor 647, Bodipy (boron dipyrromethene), in particular Bodipy 493/503, Bodipy R6G-X, Bodipy 530/550, Bodipy 558/568, Bodipy 564/570, Bodipy TMR-X, Bodipy 576/589, Bodipy 581/591, Bodipy TR-X, Bodipy 630/650, Bodipy 650/665 and Bodipy FL, Oregon Green, in particular Oregon Green 488, Oregon Green 500 and Oregon Green 514, FAM, FITC, fluoresceine, fluoresceine-dT, Rhodamine Green, TET, JOE, Yakima Yellow, HEX, CY3, TAMRA, Rhodamine Red, CY3.5, ROX, Texas Red, CY5, CY5.5, IRD700 and IRD800.
